(19) United States
(12) Patent Application Publication
(10) Pub. No.: US 2024/0165092 A1
(43) Pub. Date: May 23, 2024

(54) USE OF AN OREXIN 2 RECEPTOR AGONIST FOR POST OPERATION RECOVERY

(71) Applicant: Takeda Pharmaceutical Limited Company, Osaka (JP)

(72) Inventors: Haruhide KIMURA, Osaka (JP); Motohisa SUZUKI, Osaka (JP); James CRONICAN, Scarborough, ME (US)

(21) Appl. No.: 18/553,348

(22) PCT Filed: Apr. 1, 2022
(86) PCT No.: PCT/IB2022/053085

§ 371 Date: Sep. 29, 2023

Related U.S. Application Data
(60) Provisional application No. 63/170,211, filed on Apr. 2, 2021.

(51) Int. Cl. A61K 31/445; A61P 11/00; A61P 39/00

(52) U.S. Cl. CPC A61K 31/445; A61P 11/00; A61P 39/00

(57) ABSTRACT

A method for recovering post operation in a subject in need thereof is disclosed, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

### TECHNICAL FIELD

Certain embodiments of the present invention relate to a use of an orexin 2 receptor agonist for post operation recovery (the improvement of awareness delay after anesthesia maintenance and the improvement of sedation and respiratory depression due to opioids).

### BACKGROUND OF THE INVENTION

There is currently no drug to facilitate time to recovery from anesthesia. Adequate levels of analgesics, especially opioids, are commonly used to manage pain during postoperative period. However, opioid-related adverse effects such as sedation, respiratory depression may limit use of opioid at postoperative period (Non-Patent Document 1). Opioid antagonist such as naloxone is widely used when opioid-induced severe sedation and respiratory depression are observed. But, opioid antagonists decrease not only these adverse effects but also analgesic effect by opioids. Therefore, drugs that promote time to recovery from anesthesia, and suppress opioid-induced adverse effects with keeping analgesic effect of opioid would be needed.

Orexin system plays an important role in the control of sleep/wakefulness states (Non-Patent Document 2). There are two postsynaptic G protein-coupled receptors for orexin peptides, orexin 1 receptor (OXIR) and orexin 2 receptor (OX2R) (Non-Patent Document 3). Of the two receptors, OX2R is thought to play a pivotal role in sleep/wake regulation because OX2R KO mice, but not OX1R KO mice, show abnormal sleep cycle (Non-Patent Document 2). Methyl 3-((methylsulfonyl)amino)-2-(((4-phenylcyclohexyl)oxy)methyl)piperidine-1-carboxylate (Compound (I)) is disclosed as an orexin 2 receptor agonist (Patent Document 1).
Patent Document 1: WO 2017/135306 A1
Non-Patent Document 1: Sleep Med Rev 11 (2007) 35-46
Non-Patent Document 2: Pharmacol Rev 61 (2009) 162-176
Non-Patent Document 3: Cell 92 (1998) 573-585

### SUMMARY OF THE INVENTION

### Problems to be Solved by the Invention

Disclosed herein is a method for post operation recovery (the improvement of awareness delay after anesthesia maintenance and the improvement of sedation and respiratory depression due to opioids) in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

### Means of Solving the Problems

The present inventors have found that Compound (I) in the present specification is useful for post operation recovery (the improvement of awareness delay after anesthesia maintenance and the improvement of sedation and respiratory depression due to opioids). As a result of further studies, they have completed the present invention.

Accordingly, the present invention includes the following embodiments:

[1] A method for recovering post operation in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[2] A method for facilitating recovery or reducing a recovery time from anesthesia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[3] A method for preventing or treating postoperative respiratory disorders/depression or respiratory disorders/depression induced by opioids in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[4] A method for reducing a side effect of opioids in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[5] A method for preventing or treating postoperative sedation or sedation induced by opioids in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[6] The method of any one of [1]-[5], wherein the administration is non-oral administration.

[7] The method of [6], wherein the non-oral administration is intravenous (also referred to as IV) administration, subcutaneous (also referred to as SC) administration, transdermal administration or transmucosal administration.

In other embodiments, the present invention relates to the following:

[8] A method for accelerating emergence from anesthesia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[9] A method for preventing delayed emergence from anesthesia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[10] A method for improving pain management of a subject under pain by addressing the limiting side effect of opioids in the subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

[11] A method for improving pain management of a subject recovering post operation by addressing the limiting side effect of opioids in the subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof.

The present invention further relates to the following embodiments:

[1a] Use of Compound (I) or a salt thereof for recovering post operation.

[2b] Use of Compound (I) or a salt thereof for facilitating recovery or reducing a recovery time from anesthesia.

[3c] Use of Compound (I) or a salt thereof for preventing or treating postoperative respiratory disorders/depression or respiratory disorders/depression induced by opioids.

[4d] Use of Compound (I) or a salt thereof for reducing a side effect of opioids.

[5e] Use of Compound (I) or a salt thereof for preventing or treating postoperative sedation or sedation induced by opioids.

The present invention further relates to the following embodiments:

[1aa] A pharmaceutical composition for recovering post operation, comprising Compound (I) or a salt thereof.

[2bb] A pharmaceutical composition for facilitating recovery or reducing a recovery time from anesthesia, comprising Compound (I) or a salt thereof.

[3cc] A pharmaceutical composition for preventing or treating postoperative respiratory disorders/depression or respiratory disorders/depression induced by opioids, comprising Compound (I) or a salt thereof.

[4dd] A pharmaceutical composition for reducing a side effect of opioids, comprising Compound (I) or a salt thereof.

[5ee] A pharmaceutical composition for preventing or treating postoperative sedation or sedation induced by opioids, comprising Compound (I) or a salt thereof.

### Effect of the Invention

Compound (I) in the present specification has an orexin type 2 receptor agonist activity and is useful for post operation recovery (the improvement of awareness delay after anesthesia maintenance and the improvement of sedation and respiratory depression due to opioids).

### DETAILED DESCRIPTION OF THE INVENTION

Disclosed herein is methyl 3-((methylsulfonyl)amino)-2-(((4-phenylcyclohexyl)oxy) methyl)piperidine-1-carboxylate (Compound (I)), or a salt thereof, compositions and kits comprising Compound (I), or a salt thereof, and methods of using Compound (I), or a salt thereof.

The definition of each wording used in the present specification is described in detail in the following.

In the present specification, examples of “recovering post operation” include “facilitating recovery from anesthesia”, “reducing a recovery time from anesthesia”, “preventing or treating postoperative respiratory disorders/depression”, “preventing or treating respiratory disorders/depression induced by opioids”, “preventing or treating postoperative sedation”, and “preventing or treating sedation induced by opioids”. In some embodiments, “recovering post operation” means “post operation recovery” or “postoperative recovery”.

In some embodiments, “post operation” means “post surgery”, “after surgery” or “after surgical operations”. In some embodiments, “postoperative” means “postsurgical”.

In some embodiments, “anesthesia” is induced by anesthetics. Examples of the anesthetics include inhalation anesthetics and intravenous anesthetics. Examples of the inhalation anesthetics include isoflurane, enflurane, methoxyflurane, sevoflurane, desflurane, halothane and any optional combination thereof. Examples of the intravenous anesthetics include propofol, thiopental, thiamylal, midazolam, flumazenil, ketamine, dexmedetomidine (hydrochloride), droperidol, etomidate and any optional combination thereof. The anesthetics are used in their effective amounts for inducing anesthesia. These amounts are appropriately decided depending on the purpose or kinds of anesthetics, the administration route of anesthetics, the administration subject, and the like.

In the present specification, examples of “postoperative respiratory disorders/depression” include hypoxemia, hypoxia and hypercapnia. In some embodiments, hypoxemia is defined as peripheral oxygen saturation (SpO₂) of at most 90 percent.

In the present specification, examples of “side effect of opioids” include respiratory disorder/depression and sedation. Examples of the respiratory disorder/depression include hypoxemia, hypoxia and hypercapnia. In some embodiments, the respiratory disorder/depression is postoperative respiratory disorders/depression. In some embodiments, the sedation is postoperative sedation.

In the present specification, examples of “opioids” include fentanyl, hydromorphone, morphine, oxycodone, oxymorphone, tramadol, buprenorphine, pethidine, pentazocine, doxapram and any optional combination thereof. The opioids are used in their effective amounts for pain management or pain control during the operation (or surgery). These amounts are appropriately decided depending on the purpose or kinds of opioids, the administration route of opioids, the administration subject, and the like.

In the present specification, examples of “the side effect of opioids” include respiratory disorder/depression and sedation. Examples of the respiratory disorder/depression include hypoxia and hypercapnia. In some embodiments, the respiratory disorder/depression is postoperative respiratory disorder/depression. In some embodiments, the sedation is postoperative sedation.

In some embodiments, Compound (I) is an optically active compound. In some embodiments, Compound (I) is methyl (2R,3S)-3-((methylsulfonyl)amino)-2-(((cis-4-phenylcyclohexyl)oxy)methyl)piperidine-1-carboxylate (Compound A) in any of the methods, the uses and the pharmaceutical compositions disclosed herein. Compound (I) including its salt and its optical active compound may be produced as disclosed in WO2017/135306. In any of the uses and the pharmaceutical compositions disclosed herein, Compound (I) is used in an effective amount thereof.

In some embodiments, the effective amount is between about 3 mg to about 500 mg. In some embodiments, the effective amount is between about 5 mg to about 300 mg. In some embodiments, the effective amount is between about 5 mg to about 100 mg. In some embodiments, the effective amount is between about 5 mg to about 50 mg.

Compound (I) (hereinafter sometimes to be simply abbreviated as the compound of the present invention) can be used as it is or in the form of a pharmaceutical composition (also referred to as a medicament) by mixing with a pharmacologically acceptable carrier etc. to mammals (e.g., human, mouse, rat, rabbit, dog, cat, bovine, horse, swine, monkey).

As pharmacologically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Preferable examples of the excipient include lactose, sucrose, D-mannitol, D-sorbitol, starch, gelatinated starch, dextrin, crystalline cellulose, low-substituted hydroxypropylcellulose, sodium carboxymethylcellulose, gum arabic, pullulan, light anhydrous silicic acid, synthetic aluminum silicate and magnesium alumino metasilicate.

Preferable examples of the lubricant include magnesium stearate, calcium stearate, talc and colloidal silica.

Preferable examples of the binder include gelatinated starch, sucrose, gelatin, gum arabic, methylcellulose, carboxymethylcellulose, sodium carboxymethylcellulose, crystalline cellulose, sucrose, D-mannitol, trehalose, dextrin, pullulan, hydroxypropylcellulose, hydroxypropylmethylcellulose and polyvinylpyrrolidone.

Preferable examples of the disintegrant include lactose, sucrose, starch, carboxymethylcellulose, calcium carboxymethylcellulose, croscarmellose sodium, sodium carboxymethyl starch, light anhydrous silicic acid and low-substituted hydroxypropylcellulose.

Preferable examples of the solvent include water for injection, physiological brine, Ringer's solution, alcohol, propylene glycol, polyethylene glycol, sesame oil, corn oil, olive oil and cottonseed oil.

Preferable examples of the solubilizing agent include polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as poly(vinyl alcohol), polyvinylpyrrolidone, carboxymethylcellulose sodium, methylcellulose, hydroxymethylcellulose, hydroxyethylcellulose, hydroxypropylcellulose and the like, polysorbates; and polyoxyethylene hydrogenated castor oil.

Preferable examples of the isotonicity agent include sodium chloride, glycerol, D-mannitol, D-sorbitol and glucose.

Preferable examples of the buffer include buffers of phosphate, acetate, carbonate, citrate etc.

Preferable examples of the soothing agent include benzyl alcohol.

Preferable examples of the preservative include p-oxybenzoate esters, chlorobutanol, benzyl alcohol, phenethyl alcohol, dehydroacetic acid and sorbic acid.

Preferable examples of the antioxidant include sulfite salts and ascorbate salts.

Preferable examples of the colorant include aqueous food tar colors (e.g., food colors such as Food Color Red Nos. 2 and 3, Food Color Yellow Nos. 4 and 5, Food Color Blue Nos. 1 and 2 and the like food colors), water insoluble lake dyes (e.g., aluminum salt of the above-mentioned aqueous food tar color), natural dyes (e.g., (3-carotene, chlorophyll, red iron oxide) and the like.

Preferable examples of the sweetening agent include saccharin sodium, dipotassium glycyrrhizinate, aspartame and stevia.

Examples of the dosage form of the above-mentioned pharmaceutical composition include oral preparations such as tablet (including sugar-coated tablet, film-coated tablet, sublingual tablet, orally disintegrating tablet, buccal tablet), capsule (including soft capsule, microcapsule), pill, granule, powder, troche, syrup, liquid, emulsion, suspension, aerosol, films (e.g., orally disintegrable films, oral mucosa-adhesive film) and the like; and parenteral agents such as injection (e.g., subcutaneous injection, intravenous injection, intramuscular injection, intraperitoneal injection, drip infusion), external preparation (e.g., transdermal absorption type preparation, ointment, lotion, adhesive preparation), suppository (e.g., rectal suppository, vaginal suppository), pellet, nasal preparation, pulmonary preparation (inhalant), eye drop and the like. The compound and medicament of the present invention can be respectively safely administered orally or parenterally (e.g., intrarectal, intravenous, intraarterial, intramuscular, subcutaneous, intraorgan, intranasal, intradermal, instillation, intracerebral, intravaginal, intraperitoneal, intratumoral, proximal tumor administrations, and administration to the lesion).

These preparations may be a release control preparation (e.g., sustained-release microcapsule) such as an immediate-release preparation, a sustained-release preparation and the like.

The pharmaceutical composition can be produced according to a method conventionally used in the field of pharmaceutical formulation, for example, the method described in the Japanese Pharmacopoeia, and the like.

While the content of the compound of the present invention in the pharmaceutical composition of the present invention varies depending on the dosage form, dose of the compound of the present invention and the like, it is, for example, about 0.1 to 100 wt %.

When an oral preparation is produced, coating may be applied where necessary for the purpose of taste masking, enteric solubility or sustainability.

Examples of the coating base used for coating include sugar coating base, water-soluble film coating base, enteric film coating base, and sustained-release film coating base.

As the sugar coating base, sucrose is used, and one or more kinds selected from talc, and the precipitated calcium carbonate, gelatin, gum arabic, pullulan, carnauba wax and the like may be further used in combination.

Examples of the water-soluble film coating base include cellulose polymers such as hydroxypropylcellulose, hydroxypropylmethylcellulose, hydroxyethylcellulose, methylhydroxyethylcellulose and the like; synthetic polymers such as polyvinyl acetal diethylaminoacetate, aminoalkylmethacrylate copolymer E [Eudragit E (trade name)], polyvinylpyrrolidone and the like; and polysaccharides such as pullulan and the like.

Examples of the enteric film coating base include cellulose polymers such as hydroxypropylmethylcellulose phthalate, hydroxypropylmethylcellulose acetate succinate, carboxymethylethylcellulose, cellulose acetate phthalate and the like; acrylic acid polymers such as methacrylic acid copolymer L [Eudragit L (trade name)], methacrylic acid copolymer LD [Eudragit L-30D-55 (trade name)], methacrylic acid copolymer S [Eudragit S (trade name)] and the like; and naturally-occurring substances such as shellac and the like.

Examples of the sustained-release film coating base include cellulose polymers such as ethylcellulose and the like; and acrylic acid polymers such as aminoalkylmethacrylate copolymer RS [Eudragit RS (trade name)], ethyl acrylate-methyl methacrylate copolymer suspension [Eudragit NE (trade name)] and the like.

Two or more kinds of the above-mentioned coating bases may be used in a mixture at an appropriate ratio. In addition, for example, light shielding agents such as titanium oxide, red ferric oxide and the like may also be used during coating.

Since the compound of the present invention shows low toxicity (e.g., acute toxicity, chronic toxicity, genetic toxicity, reproductive toxicity, cardiotoxicity, carcinogenicity) and less side effects, it can be used as a prophylactic or therapeutic agent, or diagnostic agent for various diseases in mammals (e.g., human, bovine, horse, dog, cat, monkey, mouse, rat).

Moreover, the compound of the present invention is expected to be superior in central migration.

Examples of the subject to be employed in methods or uses of the present inventions include mammals such as human, bovine, horse, dog, cat, monkey, mouse and rat, with preference given to human. In some embodiments, the subject is a subject at high risk of Obstructive Sleep Apnea (OSA) or with OSA following surgery requiring general anesthesia.

While the dose of the compound of the present invention varies depending on the subject of administration, administration route, target disease, symptom and the like, for example, when the compound of the present invention is administered orally or parenterally to an adult patient, its dose is for example, about 0.01 to 100 mg/kg body weight per dose, preferably 0.1 to 50 mg/kg body weight per dose and more preferably 0.5 to 20 mg/kg body weight per dose. This amount is desirably administered in one to 3 portions daily. In some embodiments, the compound of the present invention is administered after the operation (or surgery) and after the administration of opioids as the pain controlling agent.

In some embodiments, the methods of the present invention comprise the following administration step:
administering to the subject an effective amount of anesthetics to induce anesthesia before the operation (or surgery), and then an effective amount of Compound (I) or a salt thereof after the operation (or surgery).

In some embodiments, the methods of the present invention comprise the following administration step:
administering to the subject an effective amount of anesthetics to induce anesthesia before the operation (or surgery), an effective amount of Compound (I) or a salt thereof after the operation (or surgery), and then an effective amount of opioids.

In some embodiments, the methods of the present invention comprise the following administration step:
administering to the subject an effective amount of anesthetics to induce anesthesia before the operation (or surgery), an effective amount of Compound (I) or a salt thereof after the operation (or surgery), an effective amount of opioids, and then an effective amount of Compound (I) or a salt thereof.

The compound of the present invention can be used in combination with other drugs (hereinafter to be abbreviated as concomitant drug).

By combining the compound of the present invention and a concomitant drug, a superior effect, for example,
(1) the dose can be reduced as compared to single administration of the compound of the present invention or a concomitant drug,
(2) the drug to be combined with the compound of the present invention can be selected according to the condition of patients (mild case, severe case and the like),
(3) the period of treatment can be set longer by selecting a concomitant drug having different action and mechanism from the compound of the present invention,
(4) a sustained treatment effect can be designed by selecting a concomitant drug having different action and mechanism from the compound of the present invention,
(5) a synergistic effect can be afforded by a combined use of the compound of the present invention and a concomitant drug, and the like, can be achieved.

In the present specification, the compound of the present invention and a concomitant drug used in combination are referred to as the “combination agent of the present invention”.

When using the combination agent of the present invention, the administration time of the compound of the present invention and the concomitant drug is not restricted, and the compound of the present invention or a pharmaceutical composition thereof, or the concomitant drug or a pharmaceutical composition thereof can be administered to an administration subject simultaneously, or may be administered at different times. The dosage of the concomitant drug may be determined according to the dose clinically used, and can be appropriately selected depending on an administration subject, administration route, disease, combination and the like.

The administration mode of the combination agent of the present invention and the concomitant drug is not particularly limited, and the compound of the present invention and the concomitant drug only need to be combined on administration. Examples of such administration mode include the following:
(1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like.

The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like.

For example, the content of the compound of the present invention in the combination agent of the present invention differs depending on the form of a preparation, and usually from about 0.01 to about 100 wt %, preferably from about 0.1 to about 50 wt %, further preferably from about 0.5 to about 20 wt %, based on the whole preparation.

The content of the concomitant drug in the combination agent of the present invention differs depending on the form of a preparation, and usually from about 0.01 to about 100 wt %, preferably from about 0.1 to about 50 wt %, further preferably from about 0.5 to about 20 wt %, based on the whole preparation.

The content of additives such as a carrier and the like in the combination agent of the present invention differs depending on the form of a preparation, and usually from about 1 to about 99.99 wt %, preferably from about 10 to about 90 wt %, based on the preparation.

Similar contents may be employed even when the compound of the present invention and a concomitant drug are separately formulated into preparations.

Examples of the concomitant drug include the following. A therapeutic drug for narcolepsy (e.g., methylphenidate, amphetamine, pemoline, phenelzine, protriptyline, sodium oxybate, modafinil, caffeine), antiobesity drug (amphetamine, benzfetamine, bromocriptine, bupropion, diethylpropion, exenatide, fenfluramine, liothyronine, liraglutide, mazindol, methamphetamine, octreotide, octreotide, orlistat, phendimetrazine, phendimetrazine, phenmetrazine, phentermine, Qnexa (registered trade mark), phenylpropanolamine, pramlintide, propylhexedrine, recombinant leptin, sibutramine, topiramate, zimelidine, zonisamide, Lorcaserin, metformin), acetylcholine esterase inhibitor (e.g., donepezil, rivastigmine, galanthamine, zanapezil, idebenone, tacrine), antidementia agent (e.g., memantine), inhibitor of P amyloid protein production, secretion, accumulation, aggregation and/or deposition, P secretase inhibitor (e.g., 6-(4-biphenylyl)methoxy-2-[2-(N,N-dimethylamino)ethyl]tetralin, 6-(4-biphenylyl)methoxy-2-(N,N-dimethylamino)methyltetralin, 6-(4-biphenylyl)methoxy-2-(N,N-dipropylamino)methyltetralin, 2-(N,N-dimethylamino)methyl-6-(4′-methoxybiphenyl-4-yl)methoxytetralin, 6-(4-biphenylyl)methoxy-2-[2-(N,N-diethylamino)ethyl]tetralin, 2-[2-(N,N-dimethylamino)ethyl]-6-(4′-methylbiphenyl-4-yl)methoxytetralin, 2-[2-(N,N-dimethylamino)ethyl]-6-(4′-methoxybiphenyl-4-yl)methoxytetralin, 6-(2′,4′-dimethoxybiphenyl-4-yl)methoxy-2-[2-(N,N-dimethylamino)ethyl]tetralin, 6-[4-(1,3-benzodioxol-5-yl)phenyl]methoxy-2-[2-(N,N-dimethylamino)ethyl]tetralin, 6-(3′,4′-dimethoxybiphenyl-4-yl)methoxy-2-[2-(N,N-dimethylamino)ethyl]tetralin, an optically active form thereof, a salt thereof and a hydrate thereof, OM99-2 (WO01/00663)), γ secretase inhibitor, β amyloid protein aggregation inhibitor (e.g., PTI-00703, ALZHEMED (NC-531), PPI-368 (National Publication of International Patent Application No. 11-514333), PPI-558 (National Publication of International Patent Application No. 2001-500852), SKF-74652 (Biochem. J. (1999), 340(1), 283-289)), (3 amyloid vaccine, R amyloid-degrading enzyme and the like, brain function enhancer (e.g., aniracetam, nicergoline), therapeutic drug for Parkinson's disease [(e.g., dopamine receptor agonist (e.g., L-DOPA, bromocriptine, pergolide, talipexole, pramipexole, cabergoline, amantadine), monoamine oxidase enzyme (MAO) inhibitor (e.g., deprenyl, selegiline, remacemide, riluzole), anticholinergic agent (e.g., trihexyphenidyl, biperiden), COMT inhibitor (e.g., entacapone)], therapeutic drug for amyotrophic lateral sclerosis (e.g., riluzole etc., neurotrophic factor), therapeutic drug for abnormal behavior accompanying progress of dementia, wandering and the like (e.g., sedative, anti-anxiety drug), apoptosis inhibitor (e.g., CPI-1189, IDN-6556, CEP-1347), neuronal differentiation-regenerate promoter (e.g., leteprinim, xaliproden; SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3]dioxolo[4,5-f]isoindole and an optically active form, salt or hydrate thereof), non-steroidal antiinflammatory agents (meloxicam, tenoxicam, indomethacin, ibuprofen, celecoxib, rofecoxib, aspirin, indomethacin etc.), steroid drug (dexamethasone, hexestrol, cortisone acetate etc.), disease-modifying anti-rheumatic drug (DMARDs), anti-cytokine drug (e.g., TNF inhibitor, MAP kinase inhibitor), therapeutic agent for incontinence, frequent urination (e.g., flavoxate hydrochloride, oxybutynin hydrochloride, propiverine hydrochloride), phosphodiesterase inhibitor (e.g., sildenafil(citrate)), dopamine agonist (e.g., apomorphine), antiarrhythmic drugs (e.g., mexiletine), sex hormone or a derivative thereof (e.g., progesterone, estradiol, estradiol benzoate), therapeutic agent for osteoporosis (e.g., alfacalcidol, calcitriol, elcatonin, calcitonin salmon, estriol, ipriflavone, pamidronate disodium, alendronate sodium hydrate, incadronate disodium), parathyroid hormone (PTH), calcium receptor antagonists, therapeutic drug for insomnia (e.g., benzodiazepines medicament, non-benzodiazepines medicament, melatonin agonist, orexin receptor antagonists), therapeutic drug for schizophrenia (e.g., typical antipsychotic agents such as haloperidol and the like; atypical antipsychotic agents such as clozapine, olanzapine, risperidone, aripiprazole and the like; medicament acting on metabotropic glutamate receptor or ion channel conjugated-type glutamate receptor; phosphodiesterase inhibitor), benzodiazepines medicament (chlordiazepoxide, diazepam, potassium clorazepate, lorazepam, clonazepam, alprazolam etc.), L-type calcium channel inhibitor (pregabalin etc.), tricyclic or tetracyclic antidepressant (imipramine hydrochloride, amitriptyline hydrochloride, desipramine hydrochloride, clomipramine hydrochloride etc.), selective serotonin reuptake inhibitor (fluvoxamine maleate, fluoxetine hydrochloride, citalopram hydrobromide, sertraline hydrochloride, paroxetine hydrochloride, escitalopram oxalate etc.), serotonin-noradrenaline reuptake inhibitor (venlafaxine hydrochloride, duloxetine hydrochloride, desvenlafaxine hydrochloride etc.), noradrenaline reuptake inhibitor (reboxetine mesylate etc.), mirtazapine, trazodone hydrochloride, nefazodone hydrochloride, bupropion hydrochloride, setiptiline maleate, 5-HT_{1A} agonist, (buspirone hydrochloride, tandospirone citrate, osemozotan hydrochloride etc.), 5-HT_{2A} antagonist, 5-HT_{2A} inverse agonist, 5-HT₃ antagonist (cyamemazine etc.), heart non-selective P inhibitor (propranolol hydrochloride, oxprenolol hydrochloride etc.), histamine Hi antagonist (hydroxyzine hydrochloride etc.), CRY antagonist, other antianxiety drug (meprobamate etc.), tachykinin antagonist (MK-869, saredutant etc.), medicament that acts on metabotropic glutamate receptor, CCK antagonist, β3 adrenaline antagonist (amibegron hydrochloride etc.), GAT-1 inhibitor (tiagabine hydrochloride etc.), N-type calcium channel inhibitor, carbonic anhydrase II inhibitor, NMDA glycine moiety agonist, NMDA antagonist (memantine etc.), peripheral benzodiazepine receptor agonist, vasopressin antagonist, vasopressin V1b antagonist, vasopressin V1a antagonist, phosphodiesterase inhibitor, opioid antagonist, opioid agonist, uridine, nicotinic acid receptor agonist, thyroid hormone (T3, T4), TSH, TRH, MAO inhibitor (phenelzine sulfate, tranylcypromine sulfate, moclobemide etc.), therapeutic drug for bipolar disorder (lithium carbonate, sodium valproate, lamotrigine, riluzole, felbamate etc.), cannabinoid CB1 antagonist (rimonabant etc.), FAAH inhibitor, sodium channel inhibitor, anti-ADHD drug (methylphenidate hydrochloride, methamphetamine hydrochloride etc.), therapeutic drug for alcoholism, therapeutic drug for autism, therapeutic drug for chronic fatigue syndrome, therapeutic drug for spasm, therapeutic drug for fibromyalgia syndrome, therapeutic drug for headache, therapeutic drug for quitting smoking, therapeutic drug for myasthenia gravis, therapeutic drug for cerebral infarction, therapeutic drug for mania, therapeutic drug for hypersomnia, therapeutic drug for pain, therapeutic drug for dysthymia, therapeutic drug for autonomic ataxia, therapeutic drug for male and female sexual dysfunction, therapeutic drug for migraine, therapeutic drug for pathological gambler, therapeutic drug for restless legs syndrome, therapeutic drug for substance addiction, therapeutic drug for alcohol-related syndrome, therapeutic drug for irritable bowel syndrome, therapeutic drug for lipid abnormality such as cholesterol-lowering drug (statin series (pravastatin sodium, atorvastatin, simvastatin, rosuvastatin etc.), fibrate (clofibrate etc.), squalene synthetase inhibitor), therapeutic drug for abnormal behavior or suppressant of dromomania due to dementia (sedatives, antianxiety drug etc.), therapeutic drug for diabetes, therapeutic agent for diabetic complications, therapeutic drug for hypertension, therapeutic drug for hypotension, diuretic, chemotherapeutic agent, immunotherapeutic agent, antithrombotic agent, anti-cancer agent and the like.

Two or more kinds of the above-mentioned concomitant drug may be used in a mixture at an appropriate ratio.

The compound of the present invention can also be used in combination with biologics (e.g., antibody drug, nucleic acid or nucleic acid derivative, aptamer drug, vaccine preparation), or can be used in combination with a gene therapy method and the like, or can also be used in combination with a treatment in psychiatric field without using drugs.

Examples of the treatment method in the psychiatric field without using drug include modified electroconvulsive therapy, deep brain stimulation therapy, repetitive transcranial magnetic stimulation therapy, psychotherapy including cognitive behavioral therapy and the like.

The effects of Compound (I), exemplified by “recovering post operation”, “facilitating recovery from anesthesia”, “reducing a recovery time from anesthesia”, “preventing or treating postoperative respiratory disorders/depression”, “preventing or treating respiratory disorders/depression induced by opioids”, “reducing side effect of opioids”, “preventing or treating postoperative sedation” and “preventing or treating sedation induced by opioids”, are assessed or evaluated by methods shown in Experimental Examples hereinbelow.

In some embodiments, the respiratory disorders/depression is assessed by whole body plethysmography (WBP). For WBP, respiratory parameters such as respiratory rate (RR), tidal volume (TV) and minutes volume (MV) are assessed. In some embodiments, the respiratory disorders/depression is assessed by arterial oxygen saturation (SaO₂), peripheral (or percutaneous) oxygen saturation (SpO₂), end-tidal carbon dioxide tension (PETCO₂), or any optional combination thereof.

In some embodiments, the sedation is assessed by Richmond agitation-sedation scale (RASS), Ramsay sedation scale (RSS), Modified Observer's Assessment of Alertness, Sedation Scale (MOAAS) or any optional combination thereof.

As shown in Experimental Examples hereinbelow, Compound (I) does not affect or counteract the analgesic effect of opioids. Therefore, Compound (I) is quite useful in methods of “preventing or treating respiratory disorders/depression induced by opioids”, “reducing side effect of opioids”, and/or “preventing or treating sedation induced by opioids”.

Like Compound (I), other orexin receptor agonists can be used for a method for recovering post operation in a subject. As the orexin receptor agonists, the compounds disclosed in the following documents can be used (WO2017/135306, WO2018/164191, WO2018/164192, WO2019/027003, WO2019/027058, WO2020/004536, WO2020/004537, WO2020/122092, WO2020/122093, WO2020/158958, WO2020/167701, WO2020/167706, WO2021/026047).

Among the orexin receptor agonist, the orexin 2 receptor selective agonist is preferable. The orexin 2 receptor selective agonist is the compounds which have more than 200-time orexin 2 receptor agonistic activity than the orexin 1 receptor agonistic activity. More preferably, the orexin 2 receptor selective agonist is the compounds which have more than 500-time orexin 2 receptor agonistic activity than the orexin 1 receptor agonistic activity. Furthermore preferably, the orexin 2 receptor selective agonist is the compounds which have more than 1,000-time orexin 2 receptor agonistic activity than the orexin 1 receptor agonistic activity.

In some embodiments, Compound (I) or a salt thereof is useful for enabling respiratory control and the prevention of hypoxemia in adults at high risk of Obstructive Sleep Apnea (OSA) or with OSA following surgery requiring general anesthesia (inhaled or IV).

In some embodiments, Compound (I) or a salt thereof decreases the proportion of postoperative patients experiencing hypoxemia as defined as SpO₂≤90% within 90 minutes post-initiation of Compound (I) or a salt thereof by 20% (with >70% probability) vs placebo. Here, the dose of Compound (I) ranges in some embodiments from 5.6 to 22.4 mg. In some embodiments, this dose is for IV infusion.

### Examples

The following examples are put forth so as to provide those of ordinary skill in the art with a complete disclosure and description of how to make and use the compositions, and therapeutic methods of the invention, and are not intended to limit the scope of what the inventors regard as their invention.

Compound (I) (methyl 3-((methylsulfonyl)amino)-2-(((4-phenylcyclohexyl)oxy)methyl)piperidine-1-carboxylate) was prepared according to the method described in WO 2017/135306 A1 and subjected to the following Experimental Examples.

Time to Recovery from Anesthesia (Isoflurane and Propofol)

### Experimental Example 1: Effect of Compound (I) on the Time to Recovery from Isoflurane-Induced Anesthesia Assessed by Righting Reflex in Rats

Male Sprague-Dawley rats, 8 weeks-age, were purchased from Charles river Japan. To induce and maintain isoflurane anesthesia, rats were placed in a pre-filled anesthesia induction chamber isoflurane 4%. The induction of anesthesia was defined as the rat's inability to right itself into the prone position (loss of righting reflex: LORR) after it was placed in the supine position in a cage. After confirming LORR, rats were exposed to 3% isoflurane using a facemask and maintained for 30 min. Fifteen minutes after LORR, Compound (I) was administered subcutaneously (SC) to rats in a volume of 5 mL/kg of body weight (BW). As vehicle-treated control, 10.5% (w/v) Captisol® (CyDex Pharmaceuticals, KS, USA)/1.5 mM Na₂HIPO₄ in distilled water was administered to rats. Thirty minutes after confirming LORR, isoflurane inhalation was terminated and time to recovery from LORR was recorded as duration of anesthesia. Body temperature was maintained at 37.0-37.5° C. using a heating pad during and after anesthesia. The result is shown in Table 1.

TABLE 1

| | | Duration of anesthesia (min) |
|---|---|---|
| Treatment | Dose (mg/kg) | (Mean) |
| Vehicle | 0 | 6.15 |
| Compound (I) | 0.3 | 3.92 |
| | 1.0 | 3.06 |

### Experimental Example 2: Effect of Compound (I) on the Time to Recovery from Propofol-Induced Anesthesia Assessed by Righting Reflex in Rats

Male Sprague-Dawley rats, 8 weeks-age, were purchased from Charles river Japan. To induce and maintain propofol anesthesia, a bolus dose of propofol (10 mg/kg) was administered intravenously to rats. After confirming LORR, rats were gently placed in the supine position on a heating pad to maintain body temperature at 37.0-37.5° C. Subsequently, continuous administration of propofol at 60 mg/kg/hr was performed for 30 min. Fifteen minutes after LORR, Compound (I) was administered SC to rats in a volume of 5 mL/kg of BW. As vehicle-treated control, 10.5% (w/v) Captisol®/1.5 mM Na₂HPO₄ in distilled water was administered to rats. Thirty minutes after confirming LORR, propofol infusion was terminated and time to recovery from LORR was recorded as duration of anesthesia. The result is shown in Table 2.

TABLE 2

| | | Duration of anesthesia (min) |
|---|---|---|
| Treatment | Dose (mg/kg) | (Mean) |
| Vehicle | 0 | 12.8 |
| Compound (I) | 1.0 | 11.7 |
| | 3.0 | 6.70 |
| | 10 | 6.00 |

As is clear from Table 1 and Table 2, Compound (I) reduced time to recovery from isoflurane and propofol-induced anesthesia assessed by righting reflex in rats.

Suppression of Opioid (Fentanyl)-Induced Adverse Effects without Affecting Pain

### Experimental Example 3: Effect of Compound (I) of Fentanyl (0.1 mg/kg)-Induced Sedation Assessed by Righting Reflex in Rats

Male Sprague-Dawley rats, 8 weeks-age, were purchased from Charles river Japan. In this study, LORR was used as an indicator of sedation. To induce sedation, fentanyl at 0.1 mg/kg was administered SC to rats in a volume of 2 mL/kg of BW. Ten minutes after fentanyl administration, LORR was observed in all rats, and then Compound (I) was administered SC in a volume of 5 mL/kg of BW. As vehicle-treated control, 10.5% (w/v) Captisol®/1.5 mM Na₂HIPO₄ in distilled water was administered. The time to recovery from LORR was recorded as duration of sedation.

The result is shown in Table 3. Compound (I) reduced duration of fentanyl-induced sedation assessed by righting reflex in rats.

TABLE 3

| | | Duration of sedation (min) |
|---|---|---|
| Treatment | Dose (mg/kg) | (Mean) |
| Vehicle | 0 | 88.8 |
| Compound (I) | 1.0 | 81.3 |
| | 3.0 | 67.0 |
| | 10 | 42.9 |

### Experimental Example 4: Effect of Compound (I) of Fentanyl (0.1 mg/kg)-Induced Respiratory Depression Assessed by Whole Body Plethysmography (WBP) in Rats

Male Sprague-Dawley rats, 8 weeks-age, were purchased from Charles river Japan. Respiratory function was assessed by WBP that had inflow and outflow ports for the continuous delivery of fresh room air and removal of expired carbon dioxide. Under these experimental conditions, rat was placed in the WBP chamber just after administration of fentanyl (0.1 mg/kg, SC). Respiratory parameters such as respiratory rate (RR), tidal volume (TV) and minutes volume (MV) were detected from pressure transducer, and recorded with data acquisition software (FinePoint software, Data Sciences International, Inc.). All respiratory parameters were analyzed by the individual integrated values of the 1-min measured values from 0 to 30 min after administration of fentanyl as 30-min integrated value. Compound (I) was administered SC to rats 15 min before fentanyl administration.

The result is shown in Table 4. As results, fentanyl (0.1 m/kg as vehicle group) decreased RR and MV compared to that of basal group. Under these experimental conditions, Compound (I) increased RR, TV and MV compared to that of vehicle group, suggesting that Compound (I) suppressed fentanyl-induced respiratory depression in rats.

TABLE 4

| | | | RR | TV | MV |
|---|---|---|---|---|---|
| | Compound | | (breaths/ | (mL/ | (mL/ |
| | (I) | Fentanyl | min) | stroke) | min) |
| Group | (mg/kg) | (mg/kg) | (Mean) | (Mean) | (Mean) |
| Basal | 0 | 0 | 165 | 1.21 | 180 |
| | (Vehicle) | (Vehicle) | | | |
| Vehicle | 0 | 0.1 | 128 | 1.36 | 168 |
| | (Vehicle) | | | | |
| Compound | 10 | 0.1 | 144 | 1.61 | 206 |
| (I) | | | | | |
| (10) | | | | | |

### Experimental Example 5: Effects of Compound (I) on Fentanyl-Induced Analgesic Effects Assed by Using Formalin Test in Rats

Male Sprague-Dawley rats, 8 weeks-age, were purchased from Charles river Japan. A 2.5 vol % formalin solution was SC injected in the left hind paw 30 min after Compound (I) administration, Fentanyl (0.045 mg/kg) was administered 15 min after Compound (I) administration. In the measurement of pain threshold, the number of pain behavior (flinching) in 1 min was counted at 13 points of 1, 6, 11, 16, 21, 26, 31, 36, 41, 46, 51, 56 and 61 min after formalin injection. Phase I (pain caused by direct stimulus of formalin to the peripheral nervous system) was from 1 to 11 min after formalin injection, and phase II (pain related with increased sensitivity of spinal posterior horn neurons evoked by the phase I stimulus) was from 16 to 61 min after formalin injection, the number of flinching was added up in each phase.

The result is shown in Table 5. Fentanyl (0.045 mg/kg) showed analgesic effects in formalin pain model as assessed by the reduction of total number of flinches in rats. Under these experimental conditions, Compound (I) did not counteract the analgesic effect of fentanyl in rats.

TABLE 5

| | Compound | | Phase I | Phase II |
|---|---|---|---|---|
| | (I) | Fentanyl | (No. of | (No. of |
| Group | (mg/kg) | (mg/kg) | flinching) | flinching) |
| Basal | 0 (Vehicle) | 0 (Vehicle) | 21 | 75 |
| Vehicle | 0 (Vehicle) | 0.045 | 5 | 58 |
| Compound | 3.0 | 0.045 | 5 | 52 |
| (I) | | | | |
| (3) | | | | |
| Compound | 10 | 0.045 | 4 | 45 |
| (I) | | | | |
| (10) | | | | |

### Experimental Example 6: Effects of Compound (I) on Fentanyl-Induced Analgesic Effects Assed by Using Pain Model of Postoperative Skin Incision in Rats

Male Sprague-Dawley rats, 8 weeks-age, were purchased from Charles river Japan. Under isoflurane anesthesia, a 1 cm longitudinal incision was made through the skin of the plantar aspect of the right foot, starting 0.5 cm from the edge of the heel and extending toward the toes. The exposed plantaris muscle was lifted with tweezers and a longitudinal incision was made. After returning the plantaris muscles to their original positions, the incisions in the skin were mattress-sewn at two points with sterilized needle thread (nylon, 5-0). After suturing, an antibiotic-containing ointment was applied to the surgical site. The surgical foot was disinfected with iodine and alcohol for disinfection, and sterilized gloves and sterile surgical instruments were used. The measurement of pain threshold (g) of the footpad of the right hind paw was measured using a Dynamic Plantar Aesthesiometer set so that the pressure reached 0 g to 50 g in 10 sec. The pain threshold was the average value of 3 measurements. Fentanyl was administered SC just after the skin incision, and Compound (I) was administered SC 15 min before the skin incision.

The result is shown in Table 6. Fentanyl (0.03 mg/kg) showed analgesic effects in postoperative skin incision model as measured by pain threshold (g) in rats. Under these experimental conditions, Compound (I) did not counteract the analgesic effect of fentanyl in rats.

TABLE 6

| | Compound | | |
|---|---|---|---|
| | (I) | Fentanyl | |
| Group | (mg/kg) | (mg/kg) | 1 hr after the skin incision (g) |
| Basal | 0 (Vehicle) | 0 (Vehicle) | 13.9 |
| Vehicle | 0 (Vehicle) | 0.03 | 26.1 |
| Compound | 3.0 | 0.03 | 28.5 |
| (I) | | | |
| (3) | | | |
| Compound | 10 | 0.03 | 32.2 |
| (I) | | | |
| (10) | | | |

There results suggested that Compound (I), OX2R selective agonist, has a potential to promote time to recovery from anesthesia- and suppress opioid-induced adverse effects without affecting pain.

### Formulation Example 1 (Production of Capsule)

1) Compound (I) 30 mg
2) crystalline cellulose 10 mg
3) lactose 19 mg
4) magnesium stearate 1 mg
total 60 mg
1), 2), 3) and 4) are mixed and filled in a gelatin capsule.

### Formulation Example 2 (Production of Tablet)

1) Compound (I) 30 g
2) lactose 50 g
3) cornstarch 15 g
4) calcium carboxymethylcellulose 44 g
5) magnesium stearate 1 g
1000 tablets 140 g in total

The total amount of 1), 2), 3) and 30 g of 4) are kneaded with water, vacuum dried and sieved. The sieved powder is mixed with 14 g of 4) and 1 g of 5), and the mixture is punched by a tableting machine. In this way, 1000 tablets containing 30 mg of Compound (I) per tablet are obtained.

### Formulation Example 3 (Production of Freeze-Dried Formulation for Injection)

Compound (I) (50 mg) is dissolved in the Japanese Pharmacopoeia distilled water for injection (50 ml), and the Japanese Pharmacopoeia distilled water for injection is added to 100 ml. The solution is filtered under sterile conditions, and 1 ml of the solution is filled in an injection vial under sterile conditions, and freeze-dried and sealed.

### INDUSTRIAL APPLICABILITY

Compound (I) of the present invention has an orexin type 2 agonist activity and is useful for post operation recovery (the improvement of awareness delay after anesthesia maintenance and the improvement of sedation and respiratory depression due to opioids).

What is claimed is:

1. A method for recovering post operation in a subject in need thereof, comprising administering to the subject an effective amount of methyl 3-((methylsulfonyl)amino)-2-(((4-phenylcyclohexyl)oxy)methyl)piperidine-1-carboxylate, or a salt thereof.

2. A method for facilitating recovery or reducing a recovery time from anesthesia in a subject in need thereof, comprising administering to the subject an effective amount of methyl 3-((methylsulfonyl)amino)-2-(((4-phenylcyclohexyl)oxy)methyl)piperidine-1-carboxylate, or a salt thereof.

3. A method for preventing or treating postoperative respiratory disorders/depression or respiratory disorders/depression induced by opioids in a subject in need thereof, comprising administering to the subject an effective amount of methyl 3-((methylsulfonyl)amino)-2-(((4-phenylcyclohexyl)oxy)methyl)piperidine-1-carboxylate, or a salt thereof.

4. A method for reducing a side effect of opioids in a subject in need thereof, comprising administering to the subject an effective amount of methyl 3-((methylsulfonyl)amino)-2-(((4-phenylcyclohexyl)oxy)methyl)piperidine-1-carboxylate, or a salt thereof.

5. A method for preventing or treating postoperative sedation or sedation induced by opioids in a subject in need thereof, comprising administering to the subject an effective amount of methyl 3-((methylsulfonyl)amino)-2-(((4-phenylcyclohexyl)oxy)methyl)piperidine-1-carboxylate, or a salt thereof.

6. The method of any one of claims 1-5, wherein the administration is non-oral administration.

7. The method of claim 6, wherein the non-oral administration is intravenous administration, subcutaneous administration, transdermal administration or transmucosal administration.
